(19) United States
(12) Patent Application Publication
(10) Pub. No.: US 2008/0045731 A1
(43) Pub. Date: Feb. 21, 2008

(54) Branched Biodiesels

(75) Inventor: Zongchao Zhang, Richland, WA (US)

Correspondence Address: AKZO NOBEL INC., INTELLECTUAL PROPERTY DEPARTMENT, 120 WHITE PLAINS ROAD 3RD FLOOR, TARRTOWN, NY, 10591, US

(73) Assignee: Akzo Nobel N.V., Arnhem (NL)

(21) Appl. No.: 11/630,067

(22) PCT Filed: Jun. 17, 2005
(86) PCT No.: PCT/EP05/52838

§ 371 Date: Dec. 19, 2006

Related U.S. Application Data
(60) Provisional application No. 60/581,787, filed on Jun. 22, 2004.

(51) Int. Cl. C07C 51/00

(52) U.S. Cl. 554/124

(57) ABSTRACT

The present invention relates to a process for branching fatty acids or alkyl esters thereof and the use of such branched fatty acid alkyl esters as a major component of biodiesel.

### FIELD OF THE INVENTION

The present invention generally relates to branched biodiesels and to their use as an alternative fuel source.

### BACKGROUND OF THE INVENTION

Biodiesel is the name for a variety of ester-based oxygenated fuels made from vegetable oils or animal fats. Biodiesels are typically the alkyl ester of fatty acids. The common alkyls are methyl, ethyl and isopropyl, although higher alkyl groups such as 2-ethylhexyl are also reported. The fatty acids normally used in biodiesels include soy and rapeseed.

Today's diesel engines require a clean-burning, stable fuel that performs well under a variety of operating conditions. Biodiesel is the only alternative fuel that can be used directly in any existing, unmodified diesel engine. Because it has similar properties to petroleum diesel fuel, biodiesel can be blended in any ratio with petroleum diesel fuel. Many federal and state fleet vehicles in USA are already using biodiesel blends in their existing diesel engines.

The low emissions of biodiesel make it an ideal fuel for use in marine areas, national parks and forests, and heavily polluted cities. Biodiesel has many advantages as a transport fuel. For example, biodiesel can be produced from domestically grown oilseed plants such as canola. Producing biodiesel from domestic crops reduces dependence on foreign petroleum, increases agricultural revenue, and creates jobs.

There are many advantages associated with the use of biodiesels. Biodiesel is the only alternative fuel in the US to complete EPA Tier I Health Effects Testing under section 211 (b) of the Clean Air Act, which provide the most thorough inventory of environmental and human health effects attributes that current technology will allow.

Biodiesel is the only alternative fuel that runs in any conventional, unmodified diesel engine. And, it can be stored anywhere that petroleum diesel fuel is stored.

Biodiesel can be used alone or mixed in any ratio with petroleum diesel fuel. The most common blend is a mix of 20% biodiesel with 80% petroleum diesel, or “B20.”

The lifecycle production and use of biodiesel produces approximately 80% less carbon dioxide emissions, and almost 100% less sulphur dioxide. Combustion of biodiesel alone provides over a 90% reduction in total unburned hydrocarbons, and a 75-90% reduction in aromatic hydrocarbons. Biodiesel further provides significant reductions in particulates and carbon monoxide than petroleum diesel fuel. Biodiesel provides a slight increase or decrease in nitrogen oxides depending on engine family and testing procedures. Based on Ames Mutagenicity tests, biodiesel provides a 90% reduction in cancer risks.

Biodiesel is 11% oxygen by weight and contains no sulphur. The use of biodiesel can extend the life of diesel engines because it is more lubricating than petroleum diesel fuel, while fuel consumption, auto ignition, power output, and engine torque are relatively unaffected by biodiesel.

Biodiesel is safe to handle and transport because it is as biodegradable as sugar, 10 times less toxic than table salt, and has a high flashpoint of about 125° C. compared to petroleum diesel fuel, which has a flash point of 55° C. Biodiesel can be made from domestically produced, renewable oilseed crops such as soybeans, canola, cotton seed and mustard seed.

Biodiesel is a proven fuel with over 30 million successful US road miles, and over 20 years of use in Europe.

When burned in a diesel engine, biodiesel replaces the exhaust odor of petroleum diesel with the pleasant smell of popcorn or French fries.

The production of biodiesel, or alkyl esters, is well known. There are three basic routes to ester production from oils and fats:
Base catalyzed transesterification of the oil with alcohol.
Direct acid catalyzed esterification of the oil with methanol.
Conversion of the oil to fatty acids, and then to Alkyl esters with acid catalysis.

The majority of the alkyl esters produced today are done with the base catalyzed reaction because it is the most economic for several reasons:
Low temperature (66° C., 150 F) and pressure (1.4 bar, 20 psi) processing.
High conversion (98%) with minimal side reactions and reaction time.
Direct conversion to methyl ester with no intermediate steps.
Exotic materials of construction are not necessary.

The general process reacts a fat or oil with an alcohol, like methanol, in the presence of a catalyst to produce glycerine and methyl esters or biodiesel. The methanol is charged in excess to assist in quick conversion and recovered for reuse. The catalyst is usually sodium or potassium hydroxide, which has already been mixed with the methanol.

The methyl esters of the corresponding natural fatty acid(s) are desired because of their lower viscosity and pour point in application as biodiesels. The natural fatty acids are typically with linear aliphatic hydrocarbon chains that cause high viscosity and pour point in derivative products.

The present invention generally relates to the manufacture and use of branched fatty acids and alkyl esters thereof as biodiesels. Branching the fatty chain can induce a significant drop in the viscosity and pour point of fatty esters, resulting in an improved biodiesel that is easier to formulate and use.

Commercial branched acids are not, however, naturally occurring materials.

While there are several known processes for the preparation of branched acids and alkyl esters thereof, they are plagued by poor conversion and low yields.

Accordingly, there is a need for an improved process that for the preparation of aryl branched fatty acids and alkyl esters thereof from straight chain unsaturated fatty acid feedstocks with a high conversion rate, an increased selectivity towards branched monomeric isomers and which employs a reusable catalyst.

### SUMMARY OF THE INVENTION

The present invention relates to a process for branching fatty acids or alkyl esters thereof and the use of such branched fatty acid alkyl esters as a major component of biodiesel.

### DETAILED DESCRIPTION OF THE PRESENT INVENTION

The claimed invention relates to novel biodiesels based on branched fatty and/or natural acids and/or alkyl esters thereof. “Branched fatty acids or alkyl esters thereof” means fatty acids/alkyl esters containing one or more alkyl side groups, and/or aryl groups, which are attached to the carbon chain backbone at any position. Such esters include, but are not limited to, triglycerides, diglycerides, monoglycerides, and alkylesters (branched and unbranched alkyl) of branched fatty acids. The branch in the fatty acids includes but is not limited to alkyl, aromatic, alkoxy, and the like. Examples of branched fatty acids include, but are not limited to, methyl and ethyl branched fatty acids, phenyl and tolyl branched fatty acids, acetoxy branched fatty acids and the like. The branched fatty esters can be made by isomerization, alkylation, metathesis, and/or alkoxylation of triglycerides, diglycerides, monoglycerides and/or alkylesters in the presence of catalysts or transesterification of branched triglycerides with alcohols. The esters can also be made by esterification of branched acids with alcohols.

The fatty chain of the biodiesel is mostly unsaturated and polyunsaturated because polyunsaturated chains have a lower melting point and/or pour point. The methyl esters of the corresponding natural fatty acid(s) are desired for use as biodiesels because of their lower viscosity and pour point. The natural fatty acids are typically with linear aliphatic hydrocarbon chains that cause high viscosity and pour point in derivative products.

The branched biodiesel of the present invention can be prepared either by branching the fatty acid, followed by conversion to the corresponding alkyl ester, or to the alkyl ester itself. The existing commercial biodiesels can also be converted to branched biodiesels in the presence of a catalyst.

Numerous catalyst systems/processes are known for the preparation of branched fatty acids and/or the corresponding alkyl esters thereof. Examples of preferred catalyst systems/methods are described by U.S. patent application Ser. Nos. 10/177,405; 10/339,437; and 10/412,201, which are all incorporated herein by reference.

In general, branched fatty acids or alkyl esters thereof can be prepared by contacting unsaturated linear fatty acids and/or esters thereof with at least one acidic catalyst. The acidic catalyst and/or support material is characterized in that it provides the acidic sites for the isomerization of unsaturated fatty acids. Optionally, metals can be loaded on such acidic support materials to allow for subsequent hydrogenation of the aryl branched unsaturated fatty acids to saturated ones. Examples of acidic catalysts employable in the claimed process include but are not limited to zeolites, acidic clays, molecular sieves and the like.

Acidic zeolites are a preferred acid support material. Zeolites are crystalline aluminosilicates generally represented by the formula
Mⁿ⁺_{p/n}[(AlO₂)ₚ(SiO₂)_{q(q>p)}].mH₂O
where M is a metal cation of groups IA including Hydrogen or IIA and n is the valency of this metal. Zeolites consist of a network of SiO₄ and AlO₄ tetrahedra linked together via shared oxygen atoms. Aluminum has a 3⁺ valency resulting in an excess negative charge on the AlO₄ tetrahedra, which can be compensated by cations such as H⁺. When M is hydrogen the materials are Bronsted acidic, when M is for example Cs the materials are basic. Upon heating, Bronsted acidic hydroxyls condense creating coordinately unsaturated Al, which acts as a Lewis acid site. The acid strength, acid site density and Bronsted versus Lewis acidity are determined by the level of framework aluminum. The ratio of silica/alumina can be varied for a given class of zeolites either by controlled calcination, with or without the presence of steam, optionally followed by extraction of the resulting extra framework aluminum or by chemical treatment employing for example ammonium hexafluorosilicate.

As zeolite frameworks are typically negatively charged, the charge balancing cations related to this invention include monovalent cations such as H⁺, Li⁺ and the like, divalent cations such as Mg²⁺, Zn²⁺ and the like and trivalent cations such as Ln³⁺, Y³⁺, Fe³⁺, Cr³⁺ and the like. The framework composition of the three-dimensional zeolites may contain other elements in addition to Al and Si, such as, for example, P, Ti, Zr, Mn, and the like. Although any zeolite meeting the parameters of this embodiment of the present invention can be employed, faujasite (e.g. Y zeolite), Beta zeolite, Offeretite and the like are particularly well suited for the present process. The Si/Al ratio of the zeolites can vary depending on the particular zeolite employed provided that the skilled artisan understands that a ratio which is too low will result in more by-products and a ratio which is too high will lower the activity of the zeolite. In most cases the Si/Al ratio of the zeolites is at least 2, up to 500. For example, the Si/Al ratio for Beta zeolite may be from about 5-75 while that for Y zeolite can be from 2 to about 80.

Zeolites usefully employed within the embodiments of the invention are typically acidic zeolites with or without metal ions, in addition to protons. Specific examples of zeolite structures include, but are not limited to faujasite, mordenite, USY, MFI, M or, Y and Beta types.

It is to be understood that if said acidic zeolites are not loaded with metal ions, then a separate catalyst loaded with at least one metal capable of hydrogenating the branched unsaturated fatty acids may optionally be employed.

Additionally, the acid zeolite catalyst employed in the process of the present invention must have a pore size sufficiently large so as to accommodate both the fatty acid and the aromatic. Because the arylation/isomerization takes place within the pores of the zeolite catalyst, zeolites with small apertures are not suitable as they cannot allow the entry of the fatty acid and/or the aromatic. This is in stark contrast to acidic clay catalysts where the arylation is conducted on the surface of such catalysts since they do not contain pores.

In a preferred embodiment, the zeolites employable in the context of the present invention include all those with 10-ring structures and higher-ring zeolite structure types with 1-dim and 3-dim. Examples include, but are not limited to, AEL, AFO, (and others in 10-ring, 1-dim, none), FER, (and other 10-ring, 1-dim, 1-dim), MFI, MEL, MEN, and all the higher ring zeolite types.

| | | Second biggest ring | | | | | | |
|---|---|---|---|---|---|---|---|---|
| Biggest | Channel | | 7-ring | 8 ring | | 10-ring | | 12 ring |
| ring | dimension | none | 1-dim | 1 dim | 3 dim | 1-dim | 2-dim | 1-dim |
| 6-ring | | AFG, AST, DOH, LIO, | | | | | | |
| | | LOS, LTN, MEP, MTN, | | | | | | |
| | | NON, SGT, SOD | | | | | | |
| 8-ring | 1-dim | ABW, AFT, ATN, ATV, | | AEI(i), APC(i), APD(i), ATT(i), | | | | |
| | | AWW, BIK, CAS, DDR, | | BRE(i), EDI(i), GIS(i), GOO(i), | | | | |
| | | EAB, ERI, JBW, LEV | | MER(i), MON(i), PHI(i), | | | | |
| | | | | THO(i), YUG(i) | | | | |
| | 3-dim | ANA, CHA, LTA | | NAT(i) | KFI(c), | | | |
| | | | | | PAU(c), | | | |
| | | | | | RHO(c) | | | |
| 9-ring | 1-dim | CHI | | LOV(i) | | | | |
| 10-ring | 1-dim | AEL, AFO, EUO, LAU, | | DAC(i), EPI(i), FER(i), HEU(i), | | | MFI(i) | |
| | | MTT, NES, PAR, TON | | MFS(i), STI(i) | | | | |
| | 3-dim | MEL | | WEN(i) | | | | |
| 12-ring | 1 dim | AFI, ATO, ATS, CAN, | MEI(i) | AFR(i), AFS(i), AFY(i), | | BOG(i) | | EMT(i) |
| | | LTL, MTW, ROG | | BPH(i), GME(i), MAZ(c), | | | | |
| | | | | MOR(i), OFF(i) | | | | |
| | 3-dim | FAU | | | | | | BEA(i) |
| 14-ring | 1-dim | AET, UTD-1* | | | | | | |
| 18-ring | 1-dim | VFI | | | | | | |
| 20 ring | 3-dim | | | | CLO(c) | JDF-20(i)* | | |

(i)= channels intersect
(c)= channels cross without intersecting
*= structure code not yet defined

In another embodiment, the acidic zeolite of the invention is characterized in that it comprises a material having a three dimensional pore structure wherein at least one of the channel structures has a pore size large enough to allow diffusion of the branched fatty acids and/or alkyl esters thereof. More particularly, at least one of the channel structures has a pore size large enough for the fatty acid and/or alkyl ester to enter the pore and access the internal active sites. Typically, this pore size is at least about 5.5 Å, preferably at least 6.0 Å. Catalysts of this type having a three-dimensional channel structure have higher activity and are not as readily deactivated by pore mouth blockages compared to catalysts having one and/or two dimensional channel structures.

Zeolites employable comprise a three-dimensional pore structure wherein at least one channel structure has a pore size large enough to allow diffusion of the branched fatty acids and/or alkyl esters thereof. In general, the larger the number of oxygen atoms in the ring opening, the larger the pore size of the zeolite. But this size is also determined by the structural shape of the ring. Zeolite materials having a three-dimensional channel structure and a pore size of at least about 6.0 Å can generally be employed in the process of the invention. Such pore structures having a pore size of at least about 6.0 Å generally comprise 10 and/or 12 membered rings, or even larger rings in their structures.

It is known that zeolites having a three dimensional channel structure can be formed by zeolites having one dimensional channel with certain mineral acids such as nitric acid, hydrochloric acid and the like, and/or certain organocarboxylic acids such as acetic acid and oxylic acid and the like. Other methods for generating zeolites with a three dimensional channel structure are known to the skilled artisan.

In another embodiment, the invention contemplates a process wherein mesoporous aluminosilicates are used. However, other mesoporous materials based on other materials such as those comprising transition metals and post transition metals can also be employed. Catalytic materials such as those employable in the context of the present invention are described in Angewandte Chemie Int. Ed. (7, 2001, 1258), J. Am. Chem. Soc. (123, 2001, 5014), J. Phys. Chem. (105, 2001, 7963), J. Am. Chem. Soc. (122,2000,8791), Angew. Chem. Int. Ed. (40, 2001, 1255), and Chem. Mater. (14, 2002, 1144) and in Chinese Patent Application No. 01135624.3, which are incorporated herein by reference.

Generally, the synthesis of the mesoporous aluminosilicates and aluminophosphates of the present invention involves the preparation of primary and secondary zeolite building unit precursors, which are subsequently assembled to stable mesoporous zeolites in the presence of surfactant or polymeric templates. Mesoporous zeolites derived from this invention have similar acidity, thermal and hydrothermal stability as conventional zeolites, and also have high catalytic activity.

As an example, highly ordered hexagonal mesoporous aluminosilicates (MAS-5) with uniform pore sizes were synthesized from an assembly of preformed aluminosilicate precursors with cetyltrimethylammonium bromide (CTAB) surfactant. Choice of surfactant is not a limiting feature as most quaternary ammonium salts, phosphonium salts, anionic and non-ionic surfactants, and polymers which form micellar structures in solution are effective. Other examples include, but are not limited to cetyltrimethylphosphonium, octyldecyltrimethylphosphonium, cetylpyridinium, myristyltrimethylammonium, decyltrimethylammonium, dodecyltrimethylammonium, dimethyldidodecylammonium, fatty alkylamines, fatty acids, and mixtures thereof.

The aluminosilicate precursors were obtained by heating aluminosilica gels from the aqueous hydrolysis of aluminum and silicon precursors. As previously mentioned, the present invention is not limited to Al and Si precursors, and other precursors such as certain transition metal candidates can be employed. The aluminosilicate gels are heated at 80°-400° C. for 2-10 hours. The gels had a Al₂O₃/SiO₂/TEAOH/H₂O molar ratio of 1.0/7.0-350/10.0-33.0/500-2000. Mesoporous MAS-5 shows extraordinary stability in both boiling water and steam. Additionally, temperature-programmed desorption of ammonia shows that the acidic strength of MAS-5 is much higher than that of conventional mesoporous materials and is comparable to that of microporous Beta zeolite. Analysis and testing of the materials of the present invention suggest that MAS-5 consists of both mesopores and micropores and that the pore walls of the MAS-5 contain primary and secondary structural building units similar to those of microporous zeolites. The unique structural features of the mesoporous aluminosilicates of the present invention are believed to be responsible for the observed strong acidity and high thermal stability of the mesoporous mesoporous aluminosilicates of well ordered hexagonal symmetry.

Within this embodiment of the invention, the invention is not limited to zeolites in general, or to a particular zeolite, as materials other than zeolites can be employed in conjunction with the mesoporous materials of the invention. Zeolites are, however, a preferred material to be employed with the mesoporous materials of this embodiment and the use of any known or yet to be discovered zeolites in the formation of the mesoporous materials of the present invention is included within the scope of the present invention. More particularly, using precursors of other zeolite structures, one of ordinary skill in the art could readily tailor make mesoporous zeolites containing the structural features of the particular zeolite chosen. Examples of zeolites which can be employed in the context of the present invention include, but are not limited to, zeolite A, Beta zeolite, zeolite X, zeolite Y, zeolite L, zeolite ZK-5, zeolite ZK-4, zeolite ZSM-5, zeolite ZSM-11, zeolite ZSM-12, zeolite ZSM-20, ZSM-35, zeolite ZSM-23, aluminophosphates including but not limited to VPI-5 and the like, and mixtures thereof, and/or zeolitic materials having the following framework structures: AEL, AFO, AHT, BOG, CGF, CGS, CON, DFO, FAU, FER, HEU, AFS, AFY, BEA, BPH, CLO, EMT, FAU, GME, MOR, MFI, and the like.

It is known that the aluminosilicates and/or aluminophosphates can be metal containing, or non-metal containing. Within the context of this embodiment of the invention, zeolites may contain elements such transition metals, post transition metals, Ln series and the like. Specific examples include, but are not limited to B, Ti, Ga, Zr, Ge, Va, Cr, Sb, Nb, and Y.

In still another embodiment, the catalyst is a metal ion exchanged material comprising zeolites, clays, resins, amorphous oxides, molecular sieves or their mixtures. The metal ions can be from a single metal or from multiple metals, with or without other additives. The sources of metal ions can be from any salts containing the metal ions with or without ligands. Mixed metal ions or their complexes with various ligands can be used. Ion exchange can be carried out in an aqueous phase, or in the absence of aqueous phase, e.g. solid state exchange by physically mixing the solid materials with one or more metal ion containing salts followed by calcination at elevated temperature. The ion exchange level can range from trace metal ions to 100% metal ion level based on ion exchange capacity. Ion exchange to a level over 100% of ion exchange capacity can also result in active catalysts.

As an example, acidic proton form (H⁺) zeolites, such as HZSM-5, H-Mordenite, HBeta, and HY, are known to be active for the isomerization of unsaturated fatty acids to branched fatty acids. Proton form zeolites containing group VII zero valent metals are also active catalysts as zero valent metals do not affect the overall proton concentrations in zeolites.

When positively charged protons are replaced by metal ions, the overall proton concentrations decrease. As isomerization is known to typically take place via protonated carbenium ion mechanism, the concentration and strength of proton acidity are critical for skeletal isomerization activity of proton form zeolites.

In the context of the present invention the inventors have discovered that metal ion exchanged zeolites are highly active for the skeletal isomerization of unsaturated fatty acids, even at near or over 100% ion exchange. This is particularly unexpected based on conventional wisdom as the concentration of protons is significantly reduced if not completely eliminated. It is preferred that higher valent metals be employed in the catalysts of the claimed invention. By higher valent metals it is meant that the valency of the metal(s) must be greater than zero. Most divalent and trivalent metal ions from the periodic table showed improved catalytic activity over purely proton form zeolites toward the isomerization and aryl branching of unsaturated fatty acids. The activity varies with the type of cation and the degree of ion exchange.

The higher valent metals that can be exchanged on the catalyst of this embodiment of the claimed invention are non-rare earth metals including, but not limited to: Li⁺, Cu⁺, Rh⁺, Ir⁺, Mg²⁺, Ca²⁺, Mn²⁺, Fe²⁺, Co⁺, Ni²⁺, Cu²⁺, Zn²⁺, Sr²⁺, Mo²⁺, Pd²⁺, Sn²⁺, Pt²⁺, Sc³⁺, Cr³⁺, Fe³⁺, Co³⁺, Ga³⁺, Y³⁺, Nb³⁺, Ru³⁺, Rh³⁺, Ir³⁺, Bi³⁺, Ti⁴⁺, Mn⁴⁺, Zr⁴⁺, Mo⁴⁺, Sn⁴⁺, V⁵⁺, Nb⁵⁺, Mo⁶⁺, mixtures thereof and the like.

In yet another embodiment the catalyst of the invention is a phosphated, sulfated, and/or tungstated zirconia optionally doped with at least one transition metal, rare earth metal, and the like.

In still yet another embodiment the catalyst of the invention is a heteropolyanions based solid acid, also known as acidic polyoxometallates. Heteropolyacids are acidic polyoxometallates with at least one another element in addition to W, Mo, V, Nb, Ta, or U in the anions. Typical examples of heteropolyacids include, but not limited to the following:

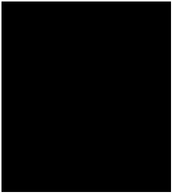

Finally, metal triflate type of lewis acid catalysts can also be usefully employed as a catalyst in the process of the present invention.

The aforementioned catalyst systems can be used to isomerize fatty acids or alkyl esters thereof to their branched counterparts, for the arylation of a fatty acid and or alkyl ester thereof into its aryl branched counterpart, for the alkoxylation of triglycerides, the transesterification of branched triglycerides with alcohols, and the esterification of branched acids with alcohols.

In one embodiment a process for the skeletal isomerization of unsaturated linear fatty acids and/or alkyl esters to their branched counterparts is envisioned. The process comprises contacting said unsaturated linear fatty acids and/or methyl esters thereof with at least one of the aforementioned catalyst systems. The catalyst and process advantageously converts fatty acid and/or alkyl ester feedstock into a mixture that is rich in branched fatty acids and/or branched alkyl esters and low in oligomers. While the reaction products of the present process will generally comprise both saturated as well as unsaturated products, both are thus included in the invention, there is high selectivity towards the formation of branched fatty acids and/or alkyl esters.

In another embodiment, the use of solid acid catalysts for the addition of aryl compounds to unsaturated fatty acids (arylation) and the isomerization of unsaturated fatty acids is contemplated. The arylation and isomerization may occur concurrently; arylation may also follow isomerization to branched unsaturated fatty acids. The balance between arylation and isomerization can be adjusted by tuning the catalyst acidity and/or reaction conditions. Under conditions employed in this invention, little cracking was observed and a small amount of lactone and ketone was formed.

The process comprises contacting unsaturated linear fatty acids and/or methyl esters thereof, and one or more aromatic compounds, with at least one solid acidic catalyst. The catalyst and process of the invention advantageously converts fatty acid and/or alkyl ester feedstock into a mixture that is rich in aryl branched fatty acids and/or aryl branched alkyl esters and low in oligomers. While the reaction products of the present process will generally comprise both saturated as well as unsaturated products, and both are thus included in the invention, there is high selectivity towards the formation of aryl branched fatty acids and/or aryl branched alkyl esters.

The invention also relates to various derivatives, primarily biodiesels, prepared from the alkyl branched and/or aryl branched fatty acids and/or alkyl esters thereof prepared in accordance with the present invention.

Good selectivity and conversion can be obtained by the process of the present invention if at least part of the isomerization or arylation is performed at a temperature of between about 100° C. and 350° C. In another embodiment, the process of the invention is performed at a temperature of between about 230° C. and 285° C. Since the conversion is also a function of the reaction/contact time, it is preferred that the fatty acid feedstock is contacted with the catalyst for a period of at least 5 minutes and reaction times of 1-16 hours are typical. An even longer period could be used if the process is operated at a lower temperature.

In general, the amount of catalyst employed in the process according to the invention is between 0.01 and 30% by weight when the process is carried out in batch or semi-batch process, based on the total reaction mixture. In another embodiment the amount of catalyst used between 0.5 and 10% by weight. In still another embodiment the catalyst amounts are between 1 and 5% by weight.

The processes of the present invention can be performed both in batch and fixed bed continuous processes. Good selectivity and conversion can be obtained by the process of the present invention if at least part of the isomerization is performed at a temperature of between about 100° C. and 350° C. In another embodiment, the process of the invention is performed at a temperature of between about 230° C. and 285° C. Since the conversion is also a function of the reaction/contact time, it is preferred that the feedstock is contacted with the catalyst for a period of at least 5 minutes and reaction times of 1-16 hours are typical. An even longer period could be used if the process is operated at a lower temperature.

When a continuous flow reactor is employed, the weight hour space velocity is between 0.01 and 100. Weight hour space velocity is defined as the weight of feed in grams passing over one gram of catalyst per hour.

Additionally, it has been found that by using a catalyst system according to this invention it is possible to reuse the catalyst. In some cases it may be desired to add fresh catalyst while optionally removing a part of the spent catalyst, and in other cases regeneration of the catalyst may be desired. Regeneration can be effected by various methods know to the skilled artisan. For example, regeneration can be accomplished by utilizing controlled oxidative regeneration and/or by washing with a solvent.

Typical feedstocks comprise fatty acids and/or esters thereof derived from natural fats and oils. Such feedstocks are predominantly unsaturated linear alkylcarboxylic acids, related esters or mixtures thereof, optionally containing other organics. Since the isomerization or conversion of unsaturated fatty acids and/or alkyl esters into their branched counterparts is desired, it is preferred to use a feedstock comprising at least about 30% by weight of said unsaturated fatty acids and/or alkyl esters thereof. In another embodiment, the feedstock comprises at least 50% by weight of unsaturated fatty acids and/or alkyl esters. Any unsaturated and/or polyunsaturated fatty acid and/or alkyl esters, or mixtures thereof is suitable as a feedstock in accordance with the present invention. In one embodiment, the feedstock comprises oleic acid as the unsaturated fatty acid and/or the alkyl ester of oleic acid in an amount of at least 40% by weight, preferably at least 70% by weight.

When it is desired to prepare an aryl branched fatty acid or alkyl ester thereof, the feedstock also comprises and/or is contacted with at least one aryl compound. Generally, the aryl compound is an aromatic that contains at least six (6) carbon atoms in the aromatic ring. Aromatic compounds are benzene and those compounds that resemble benzene in chemical behavior. For example, such compounds tend to undergo ionic substitution rather than addition and they share a similarity in electronic configuration. The aryl compound can be substituted or unsubstituted and the aromatic ring may also contain one or more heteroatoms. Preferred aryl compounds include, but are not limited to benzene, toluene, xylene, cumene, aniline, phenol, cymene, styrene, mesitylene, mixtures thereof and the like.

The products of the present invention comprise both aryl and alkyl branched fatty acids or the alkyl esters thereof. The typical ratio of aryl: branched of the products of the present invention is from about 1:1 to about 1:2.

Alkylesters/saturated fatty acids are also suited for biodiesel application as branching of these materials also dramatically reduces viscosity and pour point. Additionally, the invention contemplates all derivatives prepared from branched fatty acids and alkyl esters prepared by the processes described herein.

The aforementioned description is merely illustrative and not intended to limit the scope of the invention. Any and all of the derivatives prepared from the novel products of the present invention are within the scope of the present invention. The invention will be illustrated by the following nonlimiting examples.

### EXAMPLES

### Synthesis of Biodiesels

### Example 1

### Isomerization of Methyl Oleate in a Fixed-Bed Reactor

In a fixed bed plug flow reactor, a zeolite HBeta (powder, 1 grams) was loaded. The reaction was carried out at 250° C. at a nitrogen flow of 3 ml/min, and with the methyl oleate feed at 4.2 g/hr and 5 wt % water with respect to methyl oleate. The methyl oleate feed was converted to branched methyl oleate at over 93-95% conversion for over 7 hrs. The branched methyl oleate is suitable as biodiesel.

### Example 2

### Isomerization of Methyl Oleate in a Batch Reactor

Methyl oleate and 2 wt % of a Cu-Beta catalyst were charged into an autoclave reactor at ambient temperature. The reactor was then purged with nitrogen for three times before it was heated up to 250° C. The conversion of methyl oleate to branched methyl oleate reached 85% in 7 hours with a selectivity of 83%. The branched methyl oleate is suitable as biodiesel.

1. A biodiesel composition comprising at least one branched fatty acid ester as a base fuel, and 0 to 98% petroleum diesel.

2. The biodiesel of claim 1 wherein said branched fatty acid ester is alkyl branched, aryl branched, or mixtures thereof.

3. The biodiesel of claim 1, wherein said branched fatty acid ester is selected from the group consisting of methyl, ethyl, propyl, butyl, isopropyl, isobutyl, and/or 2-ethylhexyl esters of at said at least one fatty acid.

4. The biodiesel of claim 1 wherein said fatty acid is selected from the group consisting of rapeseed, soy, corn, sunflower, safflower, and mixtures thereof.

5. A motor fuel comprising a mixture of conventional mineral oil motor fuels and biodiesel, wherein said biodiesel comprises at least one branched fatty acid ester.

6. The biodiesel according to claim 1 wherein said branched fatty acid ester is an alkyl branched fatty acid ester prepared by:
I.) isomerizing a feedstock that comprises unsaturated linear fatty acids in the presence of at least one isomerization catalyst, in order to obtain a branched fatty acid, followed by esterification of said branched fatty acid with at least one alcohol in order to obtain an alkyl branched fatty acid ester, and/or
II.) isomerizing a feedstock that comprises fatty acid esters in the presence of at least one isomerization catalyst, in order to obtain a branched fatty acid esters, and/or
III.) isomerizing a feedstock that comprises a vegetable oil which can be used as is, or is further transesterified to an alkylester.

7. The biodiesel of claim 6 wherein said isomerization catalyst is an acidic catalys selected from the group consisting essentially of zeolites, acidic clays, molecular sieves and mixtures thereof.

8. The biodiesel of claim 7 wherein said acidic zeolite contains ring structures of at least 10 members.

9. The biodiesel of claim 8 wherein said zeolite comprises at least one of the following framework structures: AEL, AFO, AHT, BOG, CGF, CGS, CON, DFO, EUO, LAU, MTT, NES, PAR, TON, MEL, AFI, ATO, ATS, CAN, LTL, MTW, ROG, AET, UTD-1, VFI, FAU, FER, HEU, AFS, AFY, BEA, BPH, CLO, EMT, FAU, GME, MOR, MFI, MEL, MEN or mixtures thereof.

10. The biodiesel of claim 9 wherein the SiO₂/Al₂O₃ ratio of the zeolite is at least 5.

11. The biodiesel of claim 7 wherein said acidic zeolite catalyst is characterized a three-dimensional channel pore structure wherein at least one channel structure has a pore size diameter of at least 6 Å.

12. The biodiesel of claim 11 wherein said zeolite comprises at least one of the following framework structures: CON, DFO, FAU, AFS, AFY, BEA, BPH, EMT, GME, MOR, or mixtures thereof.

13. The biodiesel of claim 11 wherein said zeolite contains at least one channel structure having a pore diameter of at least 6.5 Å.

14. The biodiesel of claim 7 wherein acidic catalyst comprises a mesoporous crystalline phase having pore walls containing primary and secondary crystalline building unit structures.

15. The biodiesel of claim 14 wherein said catalyst comprises both mesopores and micropores.

16. The biodiesel of claim 15 wherein said catalyst is a mesoporous aluminosilicate or a mesoporous metal containing aluminosilicate.

17. The biodiesel of claim 16 wherein said mesoporous aluminosilicate is a mesoporous zeolite.

18. The biodiesel of claim 17 wherein said mesoporous zeolite catalyst material comprises mesopores of 15-500 Å and primary and secondary nanosized zeolite structural units in the walls that separate mesopores.

19. The biodiesel of claim 17 wherein said mesoporous zeolite comprises hexagonal mesopores, the pore wall structures of said mesopores containing primary and secondary zeolite building units.

20. The biodiesel of claim 19 wherein said primary and or secondary crystalline building units are based on at least one zeolite selected from the group consisting of zeolite A, Beta zeolite, zeolite X, zeolite Y, zeolite L, zeolite ZK-5, zeolite ZK-4, zeolite ZSM-5, zeolite ZSM 11, zeolite ZSM-12, zeolite ZSM-20, ZSM-35, zeolite ZSM-23, VPI-5 and mixtures thereof.

21. The biodiesel of claim 7 wherein said acidic catalyst comprises at least one metal ion exchanged acidic catalyst, wherein said catalyst comprises at least one non-zero valent metal ion.

22. The biodiesel of claim 21 wherein said non-zero valent metal ion is selected from the group consisting essentially of monovalent metal, divalent metal, trivalent metal, tetravalent metal, pentavalent metal, hexavalent metal and mixtures thereof.

23. The biodiesel of claim 22 wherein said higher valent metal is selected from the group consisting Li⁺, Cu⁺, Rh⁺, Ir⁺, Mg²⁺, Ca²⁺, Mn²⁺, Fe²⁺, Co⁺, Ni²⁺, Cu²⁺, Zn²⁺, Sr²⁺, Mo²⁺, Pd²⁺, Sn²⁺, Pt²⁺, Sc³⁺, Cr³⁺, Fe³⁺, Co³⁺, Ga³⁺, Y³⁺, Nb³⁺, Ru³⁺, Rh³⁺, Ir³⁺, Bi³⁺, Ti⁴⁺, Mn⁴⁺, Zr⁴⁺, Mo⁴⁺, Sn⁴⁺, V⁵⁺, Nb⁵⁺, Mo⁶⁺, mixtures thereof and the like.

24. The biodiesel of claim 21 wherein the metal ion concentration is at least 0.001% of the exchange capacity of the catalyst support.

25. The biodiesel of claim 24 wherein the metal ion concentration is at least 0.5% of the exchange capacity.

26. The biodiesel of claim 24 wherein the metal ion concentration is in the range of 0.001 to above 200% exchange level.

27. The biodiesel of claim 7 wherein said at least one catalyst is selected from the group consisting essentially of acid metal oxides comprising zirconia, niobia, silica, tungstate, or molybdates; polyoxometallates; metal triflates and mixtures thereof.

28. The biodiesel of claim 27 wherein said polyoxometallate is a heteropolyacid selected from the group consisting essentially of

| | |
|---|---|
| 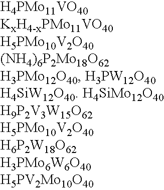 | |
| | and mixtures thereof. |

29. The biodiesel of claim 2 wherein said branched fatty acid ester is an aryl branched fatty acid ester.

30. The biodiesel of claim 29 wherein said aryl branched fatty acid ester is prepared by alkylation and isomerization of one or more aryl compounds with a feedstock which comprises unsaturated linear fatty acids, alkyl esters of unsaturated fatty acids or mixtures thereof, wherein said alkylation and isomerization is conducted in accordance with the process of claim 6.

31. The biodiesel of claim 30 wherein said aryl compound is optionally substituted with at least one heteroatom.

32. The biodiesel of claim 31 wherein said aryl compound optionally contains at least one heteroatom in its cyclic ring structure.

33. The biodiesel of claim 30 wherein said aryl compound is selected from the group consisting essentially of benzene, toluene, xylene, cumene, aniline, phenol, cymene, styrene, mesitylene, mixtures thereof and the like.
